(19) United States
(12) Patent Application Publication
(10) Pub. No.: US 2009/0157091 A1
(43) Pub. Date: Jun. 18, 2009

(54) Apparatus for Implanting Neural Stimulation Leads

(75) Inventor: John Jason Buysman, Minnetonka, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/295,850

(22) PCT Filed: Jan. 3, 2007
(86) PCT No.: PCT/US2007/000112

§ 371 Date: Oct. 9, 2008

Related U.S. Application Data
(60) Provisional application No. 60/788,980, filed on Apr. 4, 2006.

(51) Int. Cl. A61B 17/00

(52) U.S. Cl. 606/129

(57) ABSTRACT

Minimally invasive surgical instruments and procedures introducing neural stimulation leads comprise an inner member and an outer member and provide for a convenient coupling of an external stimulator lead at the inner member handle for application of test stimuli to nerves and tissue proximate the distal end of the inner member. A conductive inner member shaft proximal end extends into or proximally through the non-conductive handle and is configured to provide an inner member connector within or extending proximally to the handle for connection with a test stimulator. The inner member shaft is electrically conductive to conduct such test stimuli to nerves and tissue proximate the exposed shaft distal end. The outer member is preferably non-conductive and may thereby electrically insulate the shaft body proximal to the exposed shaft distal end. Ergonomically shaped inner member handles, caps, and inner member-outer member interlocking mechanisms are disclosed.

A wide variety of implantable electrical medical leads for conducting electrical stimuli to body tissues, nerves and organs are well known in the art. Such electrical medical leads are coupled to implantable stimulators that generate electrical stimulation pulses conducted through the lead conductor to a distal stimulation electrode or electrodes. In the field of neural stimulation or neuromodulation, neural stimulation leads are implanted in the body to dispose distal stimulation electrode(s) in operative relation to a variety of stimulation sites including in the epidural space or adjacent particular nerves for pain amelioration or in relation to organs or nerves enervating organs to apply functional electrical stimulation to elicit a body function or response. For example, neural stimulation leads are routed into the sacral region through a sacral foramen to dispose the stimulation electrodes in relation to a particular sacral nerve traversing the foramen to effect a functional response of an organ, e.g., the bladder or bowel to control voiding or genitalia to ameliorate a sexual dysfunction.

In the implantation process, it is necessary to create a tissue pathway to extend the neural stimulation lead body from the subcutaneous site of implantation of the neural stimulator to the site of stimulation. A wide variety of minimally invasive lead introducers and methods of using same have been developed to facilitate the pathway creation. Such introducers typically comprise combinations of sheaths or cannulae, penetrating needles, dilators, and obturators or stylets and/or guidewires extended through penetrating needle lumens. The neural lead is typically advanced through a needle or sheath or dilator lumen either by itself and/or over a guidewire extended through the lumen or extending through the pathway following retraction of the other introducer components.

For example, an epidural space introducer system is described in U.S. Pat. No. 4,512,351 that includes an inner stylet or obturator fitting into the needle lumen to form a Tuohy needle assembly that is extended through the lumen of an outer splittable introducer sheath or cannula. The introducer system is inserted percutaneously to reach the epidural space, the needle assembly is removed, the permanent lead is advanced through the splittable sheath lumen, and the cannula is retracted and split away from the lead body so that its connector may be coupled to an implantable pulse generator. A similar Tuohy needle assembly is described in U.S. Pat. No. 5,255,691 having interlocking needle and obturator hubs for use without an outer sheath.

In the implantation process, it is necessary to test the efficacy of the applied stimulation by coupling the neural lead connector to an external stimulator, applying stimulation, observing the results, adjusting the electrode position or selection of electrodes and the stimulation parameters, and repeating the process until the desired response is achieved. In certain systems, e.g., as described in U.S. Pat. No. 6,104,960, temporary neural stimulation leads are implanted and extended through the skin to a patient-worn external stimulator that provides stimulation for a period of days or weeks to determine if a selected stimulation regimen is efficacious. If a selected stimulation proves efficacious, a permanently implanted neural stimulation lead is implanted in the pathway and coupled to a subcutaneously implanted pulse generator.

It has also been found desirable to test for a response employing the introducer to ascertain that its distal end components are being directed through body tissue and into the epidural space or through a sacral foramen. For example, such testing is described in U.S. Pat. Nos. 6,055,456, 6,104,960, 6,512,958, 6,847,849, and 6,971,393. In the described introducer systems, the hollow needle body or shaft is made of a conductive metal having an electrically insulating coating or sheath extending along its length except in a proximal region adjacent the proximal hub and at the distal tip. One embodiment of the '958 patent further includes a dilator that is used to dilate the pathway to the sacral nerve site. The dilator also comprises a hollow conductive needle and insulating dilating sheath, whereby stimulation testing is also possible through the needle body to expose the needle distal end. An external stimulator may be coupled to the proximal region to deliver stimulation pulses through the insulated needle or dilator body to the exposed distal tip functioning as a test stimulation electrode to guide the needle tip toward the sacral nerve.

Notwithstanding these advances, a need remains for a simple, minimally invasive introducers and procedures for accessing neural stimulation sites and placing neural stimulation electrodes at such sites.

The preferred embodiments of the present invention incorporate a number of inventive features that address the above-described problems that may be combined as illustrated by the preferred embodiments or advantageously separately employed.

In preferred embodiments, an introducer (or introducer system) comprises an elongated inner member having a proximal handle and a shaft extending from the handle and sized to be disposed within a lumen of an elongated outer member to expose the shaft distal end extending distally from the outer member distal end when the outer member proximal end abuts the handle. Thus, in use, the inner member is disposed in the outer member lumen with the inner member handle extending proximally from the outer member proximal end. The so assembled introducer is advanced through body tissue to dispose the shaft distal end proximate nerves or tissue to be stimulated by electrode(s) of a neural stimulation lead.

In variations of the preferred embodiments, the inner member may comprise or function as one of a stiffening stylet, an obturator, a solid shaft needle, and a hollow core needle adapted to enable advancement of a guide wire through the needle lumen. The outer member may comprise or function as one or more of an introducer sheath or cannula or a dilator having an outer member body lumen sized to enable advancement of a neural stimulation lead through it by itself or over a guidewire introduced through it. The elongated outer member body or sheath may be splittable along its length to facilitate its withdrawal from a tissue pathway after advancement of the neural stimulation lead and placement of the lead electrode(s) in operative relation to the target nerve or tissue.

In accordance with one aspect of the invention, the introducer (and methods of using same) advantageously provides for a convenient coupling of an external stimulator lead at the inner member handle for application of test stimuli to nerves and tissue proximate the distal end of the inner member. The conductive inner member shaft proximal end extends into or proximally through the non-conductive handle and is configured to provide an inner member connector within or extending proximally to the handle for connection with a test stimulator. The inner member shaft is electrically conductive to conduct such test stimuli to nerves and tissue proximate the exposed shaft distal end. The outer member is preferably non-conductive and may thereby electrically insulate the shaft body proximal to the exposed shaft distal end.

In accordance with a further aspect of the invention, the inner member handle is preferably ergonomically shaped with gripping surfaces to be gripped with one hand to advantageously facilitate directional control of advancement of the shaft and outer member body through tissue. The inner member handle is preferably shaped having opposed major surface area sides joined by smaller surface area sides and ends. In one preferred embodiment, the opposed major surface area sides are shaped with complementary, generally concave surfaces adapted to be gripped between the fingers. Gripping surfaces may be textured or contoured to enhance gripping and reduce the possibility of slipping.

The various embodiments of the inner member connector are advantageously configured to avoid interfering with gripping the inner member handle and applying directional control to the inner member shaft and outer member sheath.

In still further embodiments, the inner member handle and the outer member sheath are advantageously coupled together and decoupled through manipulation of interlocking members at the inner member handle and the outer member proximal end.

In a variant of the invention, the inner member connector may alternatively comprise an electrical connector supported in or by the inner member handle that is coupled to the inner member shaft by electrical conductors encased within the electrically insulating handle.

Advantageously, the minimally invasive introducer embodiments and procedures minimize patient trauma and procedure time while ensuring safe and reliable introduction of neural stimulation leads.

These and other advantages and features of the present invention will be more readily understood from the following detailed description of the preferred embodiments thereof, when considered in conjunction with the drawings, in which like reference numerals indicate identical structures throughout the several views, and wherein:

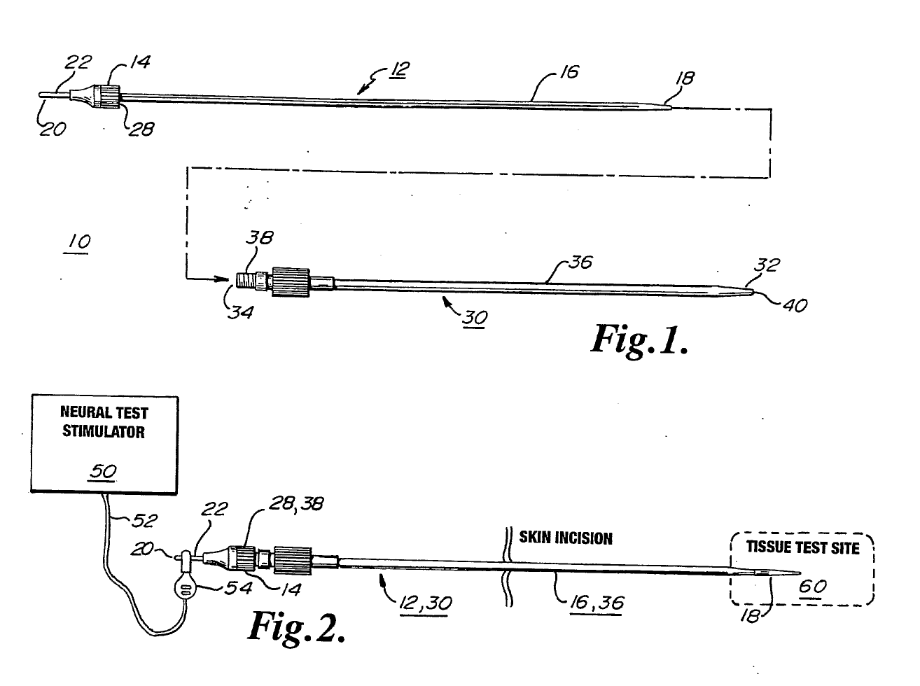

FIG. 1 is a side view of one embodiment of an introducer for a neural stimulation lead comprising an inner member having an ergonomically shaped handle and an outer member, the inner member shaft positioned to be disposed at least partly through an outer member lumen in accordance with one embodiment of the present invention;

FIG. 2 is a plan view of the inner member shaft of the inner member of FIG. 1 disposed through the outer member lumen of the outer member of FIG. 1, whereby a distal tip of the inner member shaft is electrically exposed for delivery of test stimuli by an external stimulator adapted to be coupled to an inner member connector extension of the inner member shaft extending proximally of the inner member handle;

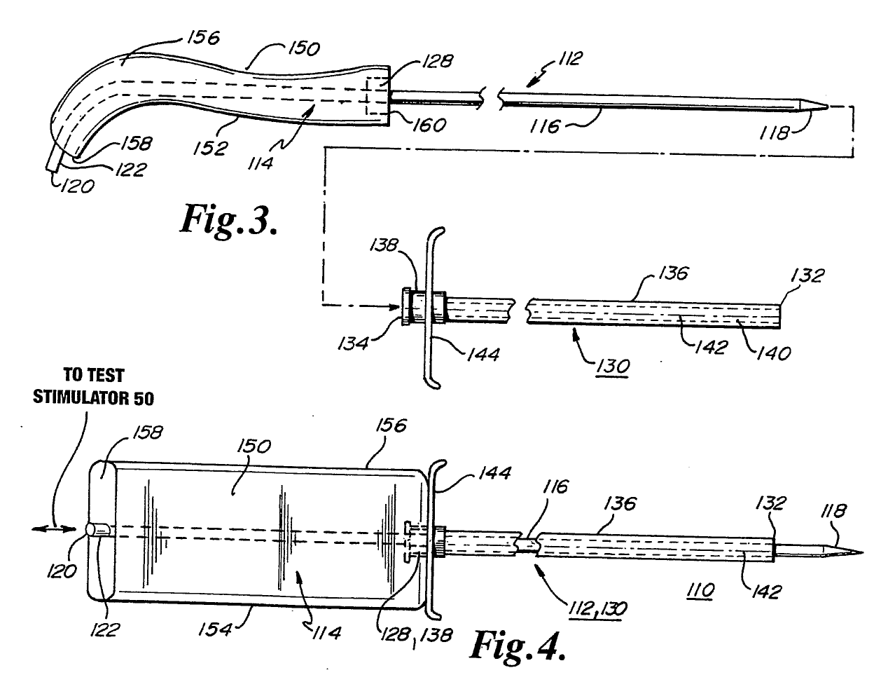

FIG. 3 is a side view of another embodiment of an introducer for a neural stimulation lead comprising an inner member having an inner member lumen extending though an inner member shaft and an outer member, the inner member shaft positioned to be disposed at least partly through an outer member lumen in accordance with a further embodiment of the present invention, the outer member having perforations enabling splitting or tearing the outer member apart;

FIG. 4 is a plan view of the inner member shaft of the inner member of FIG. 3 disposed through the outer member lumen of the outer member of FIG. 3, whereby a distal tip of the inner member shaft is electrically exposed for delivery of test stimuli by an external stimulator adapted to be coupled to an inner member connector extension of the inner member shaft extending proximally of the inner member handle;

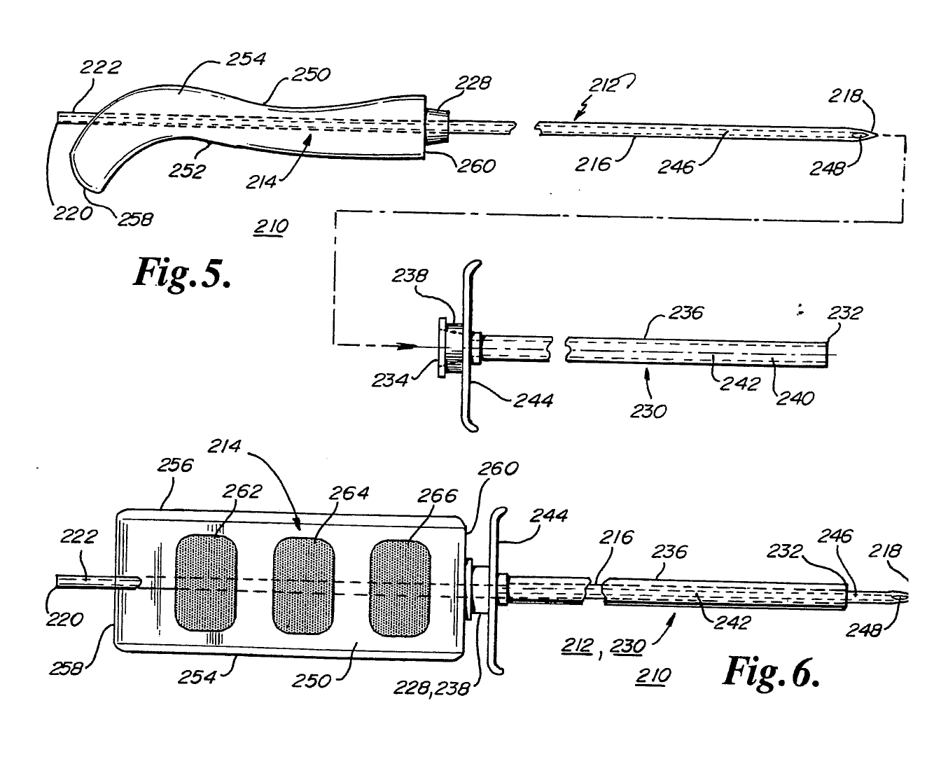

FIG. 5 is a side view of another embodiment of an introducer for a neural stimulation lead comprising an inner member having an inner member lumen extending though an inner member shaft and an outer member, the inner member shaft positioned to be disposed at least partly through an outer member lumen in accordance with a further embodiment of the present invention;

FIG. 6 is a plan view of the inner member shaft of the inner member of FIG. 3 disposed through the outer member lumen of the outer member of FIG. 3, whereby a distal tip of the inner member shaft is electrically exposed for delivery of test stimuli by an external stimulator adapted to be coupled to an inner member connector extension of the shaft extending proximally of the inner member handle, whereby a fluid and/or a stylet or guidewire may be extended through the inner member lumen;

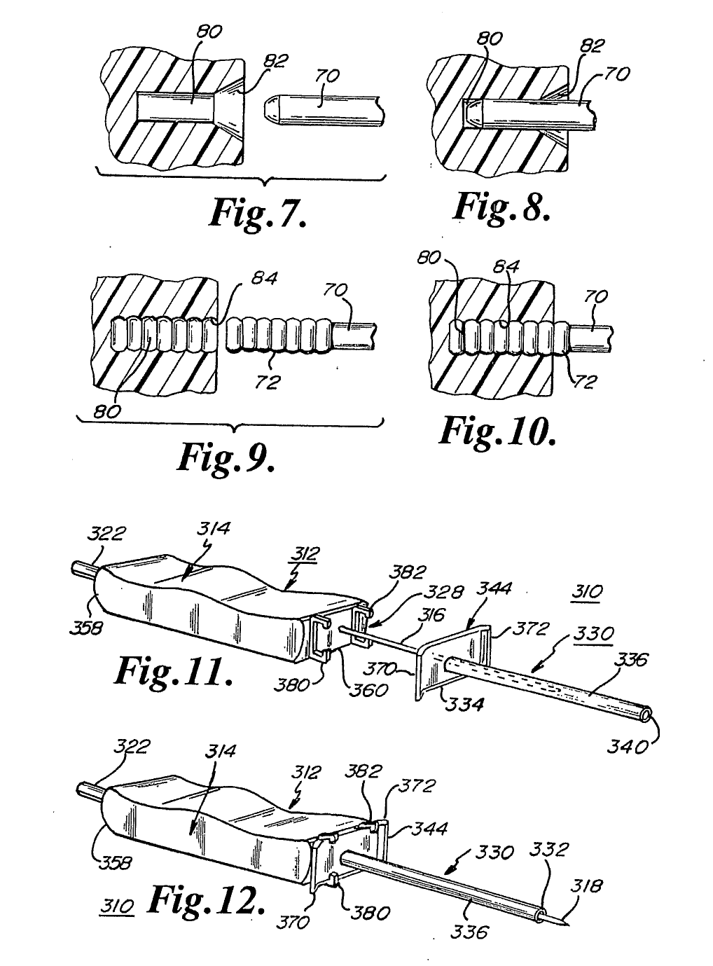

FIGS. 7 and 8 are detail views in partial cross-section of one form of frictionally interlocking the outer member proximal end with the handle of the inner member;

FIGS. 9 and 10 are detail views in partial cross-section of a further form of frictionally interlocking the outer member proximal end with the handle of the inner member;

FIG. 11 is a perspective view of an introducer for a neural stimulation lead comprising an inner member and an outer member, the outer member positioned to be disposed over the inner member shaft and locked to the inner member handle employing clips in accordance with a further embodiment of the present invention;

FIG. 12 is a perspective view of the introducer of FIG. 11 showing the outer member proximal end clipped to the handle with the inner member shaft extending through the outer member lumen to electrically expose a distal tip of the inner member shaft for delivery of test stimuli by an external stimulator adapted to be coupled to an inner member connector extension of the inner member shaft extending proximally of the inner member handle;

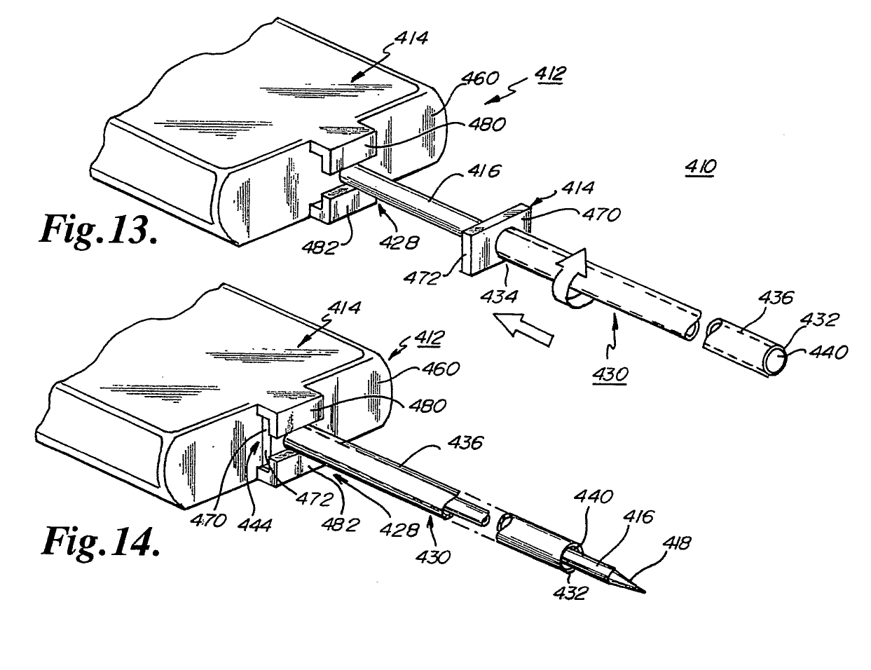

FIG. 13 is a partial perspective view of a further introducer for a neural stimulation lead comprising an inner member and an outer member having an alternative interlock mechanism for interlocking an outer member proximal end with the inner member handle;

FIG. 14 is a perspective view of the introducer of FIG. 13 showing the outer member proximal end locked to the handle with the inner member shaft extending through the outer member lumen to electrically expose a distal tip of the inner member shaft for delivery of test stimuli by an external stimulator adapted to be coupled to an inner member connector extension of the inner member shaft extending proximally of the inner member handle;

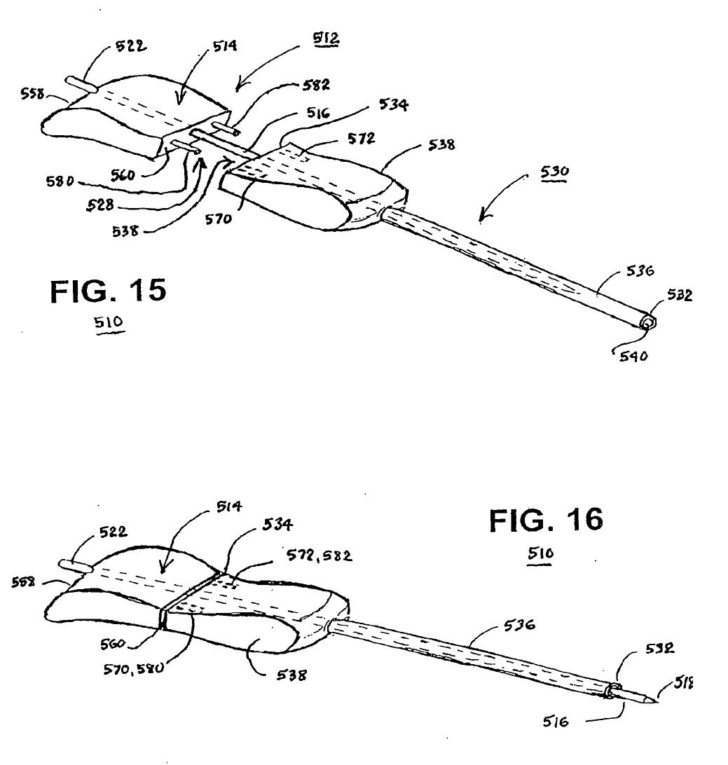

FIG. 15 is a partial perspective view of a further introducer for a neural stimulation lead comprising an inner member and an outer member having an alternative interlock mechanism for interlocking an outer member proximal end with the inner member handle invention, wherein the outer member proximal end is shaped to conform to and interlock with the inner member handle to form a distal extension of the ergonomically shaped inner member handle;

FIG. 16 is a perspective view of the introducer of FIG. 15 showing the outer member proximal end locked to the handle with the inner member shaft extending through the outer member lumen to electrically expose a distal tip of the inner member shaft for delivery of test stimuli by an external stimulator adapted to be coupled to an inner member connector extension of the inner member shaft extending proximally of the inner member handle;

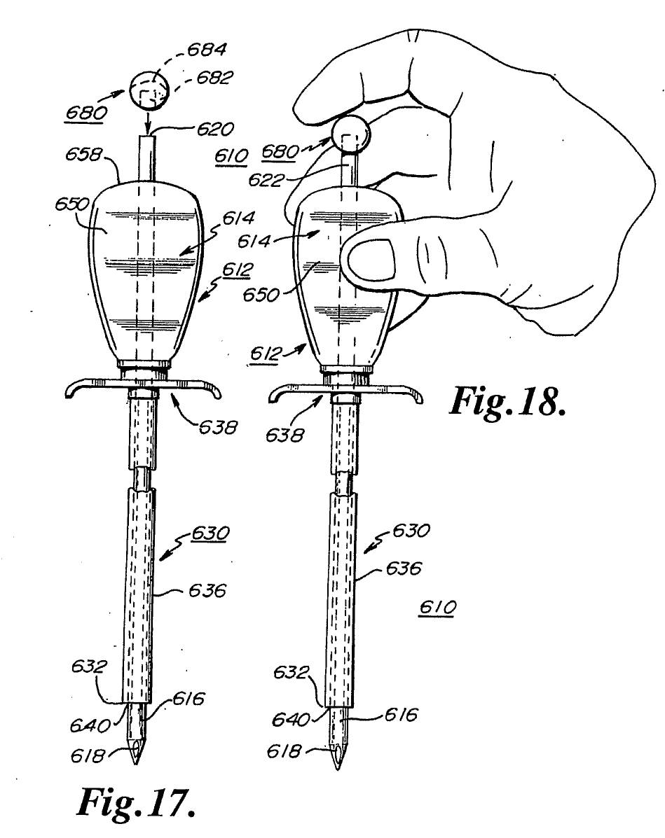

FIG. 17 is a plan view of a further embodiment of an inner member adapted to employ any interlocking mechanism with an outer member to function as an introducer of the present invention, wherein a resilient protective cap is placed over the connector extension of the inner member shaft extending proximally of the inner member handle to blunt the inner member needle proximal end;

FIG. 18 is a plan view illustrating the manipulation of the inner member handle of FIG. 17 with the protective cap in place during advancement of the inner member and an outer member through tissue;

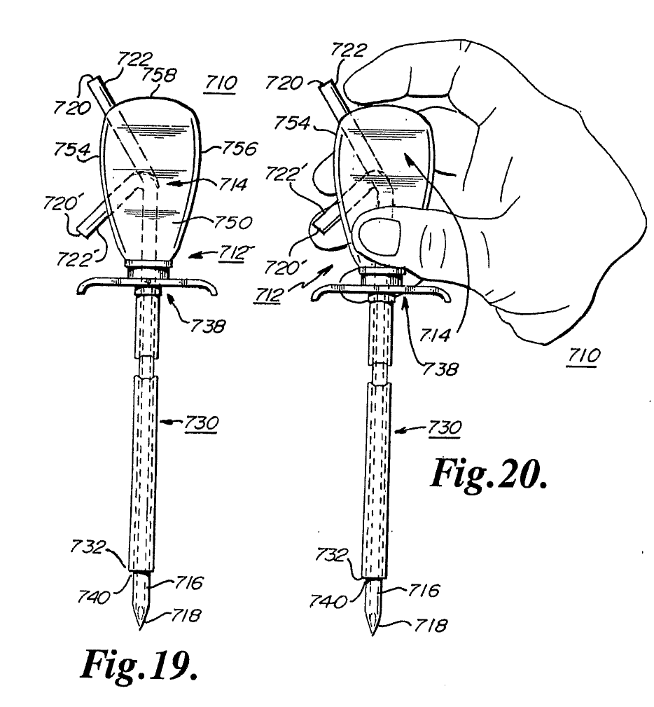

FIG. 19 is a plan view of a further embodiment of an inner member adapted to employ any interlocking mechanism with an outer member to function as an introducer of the present invention, wherein the connector extension of the inner member shaft extends toward or through the side of the inner member handle for connection to a cable extending from an external test stimulator;

FIG. 20 is a partial plan view of the grasping and manipulation of the inner member handle avoiding contact with the inner member connector of FIG. 19;

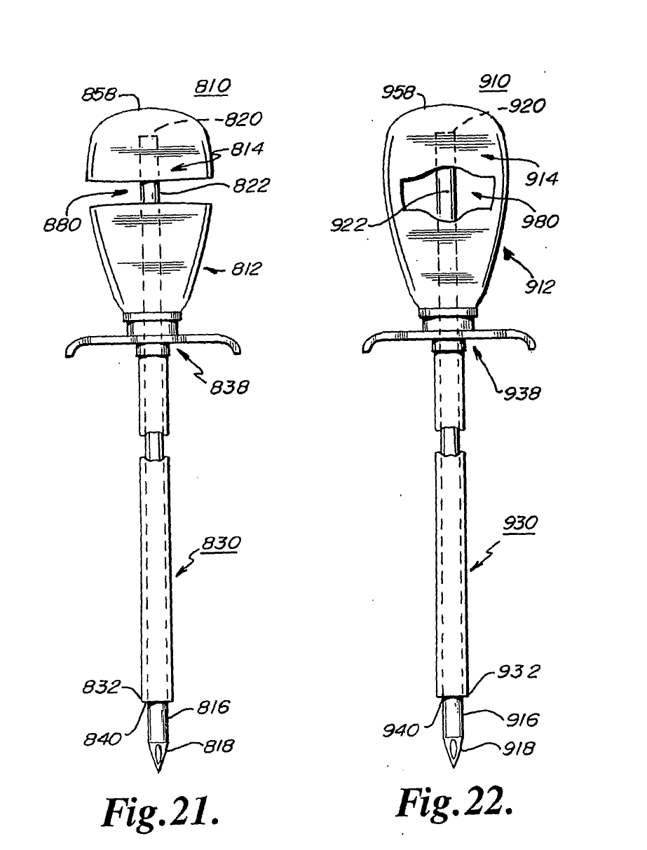

FIG. 21 is a plan view of a still further embodiment of an inner member adapted to employ any interlocking mechanism with an outer member to function as an introducer of the present invention, wherein the inner member connector extension of the inner member shaft is exposed through a window of the inner member handle for connection to a cable extending from an external test stimulator;

FIG. 22 is a plan view of a still further embodiment of an inner member adapted to employ any interlocking mechanism with an outer member to function as an introducer of the present invention, wherein the inner member connector extension of the inner member shaft is exposed through a slot extending across the inner member handle for connection to a cable extending from an external test stimulator;

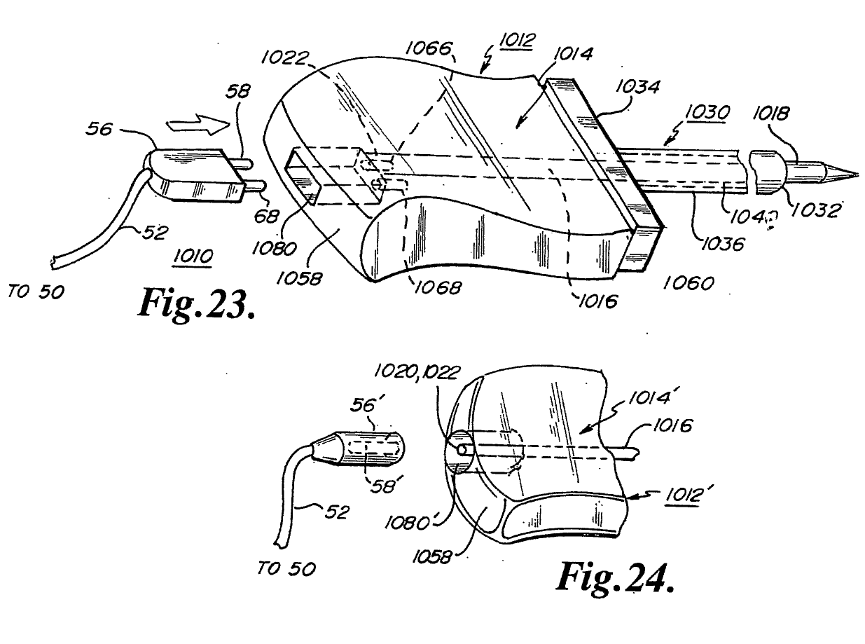

FIG. 23 is a partial perspective view of further introducer incorporating a further embodiment of a handle connector coupled to an inner member shaft that may be incorporated into the inner member handle of any of the embodiments of the inner and outer members; and

FIG. 24 is a partial perspective view of a still further embodiment of a handle connector coupled to an inner member shaft that may be incorporated into the inner member handle of any of the embodiments of the inner and outer members.

It will be understood that the drawing figures are not necessarily to scale.

In the following detailed description, references are made to illustrative embodiments of methods and apparatus for carrying out the invention. It is understood that other embodiments can be utilized without departing from the scope of the invention. Preferred embodiments for minimally invasive surgical instruments and procedures for implanting neural stimulation leads are described. It will be understood that various features and embodiments of the invention may find utility in introducers for other electrical medical leads and drug deliver catheters or other elongated medical devices.

The introducer embodiments and the methods of using same described below advantageously provide for a convenient coupling of an external stimulator lead at the inner member handle for application of test stimuli to nerves and tissue proximate the distal end of the inner member. The conductive inner member shaft proximal end extends into or proximally through the non-conductive handle and is configured to provide an inner member connector within or extending proximally to the handle for connection with a test stimulator. The inner member shaft is electrically conductive to conduct such test stimuli to nerves and tissue proximate the exposed shaft distal end. The outer member is preferably non-conductive and may thereby electrically insulate the shaft body proximal to the exposed shaft distal end.

In a first preferred embodiment depicted in FIGS. 1 and 2, an introducer 10 (or introducer system) comprises an elongated inner member 12 that is adapted to be coupled with an elongated outer member 30, inserted through a skin incision and electrically coupled to a neural test stimulator 50 for application of test stimuli to a tissue test site 60. An electrical medical lead is placed through a lumen of the outer member 30 after a suitable stimulation response is elicited at the tissue test site 60.

In variations of the preferred embodiments, the inner member 12 may comprise or function as one of a stiffening stylet, an obturator, a solid shaft needle, and a hollow core needle adapted to enable advancement of a guide wire through the needle lumen. The outer member 30 may comprise or function as one or more of an introducer sheath or cannula or a dilator having an outer member body lumen sized to enable advancement of a neural stimulation lead through it by itself or over a guidewire introduced through it. The elongated outer member body or sheath may be splittable along its length to facilitate its withdrawal from a tissue pathway after advancement of the neural stimulation lead and placement of the lead electrode(s) in operative relation to the target nerve or tissue.

The inner member 12 of FIGS. 1 and 2 comprises a proximal handle 14 and an elongated shaft 16 extending from a shaft distal end 18 through a shaft length to a shaft proximal end 20 coupled to and extending proximally from handle 14. In this embodiment, the shaft 16 comprises an elongated, electrically conductive, solid needle having a tissue penetrating tip at the shaft distal end although it will be understood that the shaft 16 may take other forms as noted above and may be only partly conductive along its length. The handle 14 is preferably non-conductive or has a non-conductive exterior surface coating. An electrically insulating coating may optionally be applied along an intermediate portion of the shaft 16 extending from a point near the shaft distal end 18 to the handle 14.

The outer member 30 of FIGS. 1 and 2 extends from an sheath distal end 32 through an outer length to a sheath or outer member proximal end 34 and is formed of an elongated sheath 36 coupled with a proximal sheath interlocking member 38 at the outer member proximal end 34. The sheath 36 may extend through the proximal sheath interlocking member 38 to or near the outer member proximal end 34. The elongated sheath 36 encloses an outer member or sheath lumen 40 extending between the outer member distal and proximal ends 32 and 34. In this embodiment, the sheath 36 is formed of an elongated, thin wall, electrically nonconductive, tubing that may or may not be tapered at the sheath distal end 32 to effect dilation of tissue that it is advanced through. It will be understood that the outer member 30 may take other forms as noted above and may be splittable along its length to facilitate removal from an electrical medical lead extending through the sheath lumen 40.

As shown in FIG. 2, the inner member shaft 16 is sized in relation to the sheath lumen 40 so that it may be extended therethrough, until the outer member proximal end 34 abuts the handle 14, to form the introducer 10. When so assembled, the pointed shaft distal end 18 extends distally from the sheath distal end 32. In use, the handle 14 and the proximal sheath interlocking member 38 are grasped and manipulated to advance the nested shaft 16 and sheath 36 through a tissue pathway from a skin incision to dispose the exposed shaft distal end at the tissue test site 60. Any of the insertion pathways disclosed in the prior art, particularly for placement of neural stimulation leads, may be followed.

The shaft proximal end 20 is electrically coupled to or forms an electrical connector 22 extending proximally from (in this embodiment) the nonconductive handle 14. In this embodiment, the connector 22 may simply be the electrically conductive exterior circumferential surface of the shaft 16 extending proximally from the handle 14 to the shaft proximal end 20. As shown in FIG. 2, an electrical cable 52 may be provided extending from the pulse generator output circuitry of the external neural test stimulator 50 to a cable connector 54, e.g., an alligator clip type connector, that may be attached to the connector 22 during application of the test stimuli. The cable connector 54 may alternatively comprise a tubular socket that axially fits over the electrical connector 22. The test stimuli are conducted through the length of the inner member shaft 16 and applied to the tissue at the tissue test site 60 by the electrically conductive exposed shaft distal end 18 in contact therewith.

In the embodiment of FIGS. 1 and 2, the inner member handle 14 and the outer member sheath 36 are advantageously coupled together and decoupled through manipulation of interlocking members at the inner member handle 14 and the outer member proximal end 34. In this embodiment, the outer member interlocking member 38 preferably comprises a male luer lock, and the inner member handle 14 incorporates a female luer lock handle interlocking member 28 that receives and interlocks with the male luer lock interlocking member 38. It will be understood that any type of conical, threaded, L-shaped slot and pin or other interlocking luer locks may be employed. The locking of interlocking members 28 and 38 advantageously prevents relative movement and slippage of the inner and outer members during passage through tissue in the selected body pathway.

Another embodiment of an introducer 110 for a neural stimulation lead is depicted in FIGS. 3 and 4 and comprises an inner member 112 and an outer member 130.

The inner member 112 comprises a proximal handle 114 and an elongated shaft 116 extending from a shaft distal end 118 through a shaft length to a shaft proximal end 120 coupled to and extending proximally from handle 114. In this embodiment, the shaft 116 comprises an elongated, electrically conductive, solid needle having a tissue penetrating tip at the shaft distal end 118 although it will be understood that the shaft 116 may take other forms as noted above and may be only partly conductive along its length. The handle 114 is preferably non-conductive or has a non-conductive exterior surface coating over the gripping surfaces. An electrically insulating coating may optionally be applied along an intermediate portion of the shaft 116 extending from a point near the shaft distal end 118 to the handle 114.

The outer member 130 of FIGS. 3 and 4 extends from a sheath distal end 132 through an outer length to a sheath or outer member proximal end 134 and is formed of an elongated sheath 136 coupled with a proximal sheath or outer member interlocking member 138 at the outer member proximal end 134. The sheath 136 may extend through the proximal sheath interlocking member 138 to or near the outer member proximal end 134. The elongated sheath 132 encloses an outer member or sheath lumen 140 extending between the outer member distal and proximal ends 132 and 134. In this embodiment, the sheath 136 is formed of an elongated, thin wall, electrically nonconductive, tubing that may or may not be tapered at the sheath distal end 132 to effect dilation of tissue that it is advanced through. A wing 144 extends across the sheath interlocking member 138, and two lines of perforations 142, disposed 180° apart around the circumference, extend along the length of the outer member 130 between the outer member distal and proximal ends 132 and 134. The lines of perforation weaken the outer member 130 so that it is splittable or capable of being torn apart along its length by grasping the opposed extensions of the wing 144 and pulling to split the outer member 130 into two parts along the lines 142 to facilitate removal from an electrical medical lead extending through the sheath lumen 140.

As shown in FIGS. 3 and 4, the inner member handle 114 is preferably ergonomically shaped to be gripped with one hand to advantageously facilitate directional control of advancement of the shaft and outer member body through tissue. The inner member handle 114 is shaped having gripping surfaces comprising opposed major surface area sides 150 and 152 joined by smaller surface area sides 154 and 156 and proximal and distal ends 158 and 160. The opposed major surface area sides 150 and 152 are shaped with complementary, generally concave surfaces adapted to be gripped between the fingers.

The shaft proximal end 120 is electrically coupled to or forms an electrical connector 122 extending proximally from (in this embodiment) the nonconductive handle 114 through handle proximal end 158. In this embodiment, the section of the shaft 116 is curved at least in part to extend within the inner member handle 114 from the handle distal end 160 to exit through the handle proximal end 158.

Again, the electrical connector 122 may simply be the electrically conductive exterior circumferential surface of the shaft 116 extending proximally from the handle proximal end 158 to the shaft proximal end 120. As shown in FIG. 2, an electrical cable 52 may be provided extending from the pulse generator output circuitry of the external neural test stimulator 50 to a cable connector 54, e.g., an alligator clip type connector, that may be attached to the connector 122 during application of the test stimuli. The test stimuli are conducted through the length of the inner member shaft 116 and applied to the tissue at the tissue test site 160 by the electrically conductive exposed shaft distal end 118 in contact therewith.

As shown in FIG. 4, the inner member shaft 116 is sized in relation to the sheath lumen 140 so that it may be extended therethrough, until the outer member proximal end 134 abuts the handle 114, to form the introducer 110. The inner member handle 114 and the outer member sheath 136 are advantageously coupled together and decoupled through manipulation of interlocking members at the inner member handle 114 and the outer member proximal end 134. In this embodiment, the outer member interlocking member 138 preferably comprises a male luer lock, and the inner member handle 114 incorporates a female luer lock handle interlocking member 128 that receives and interlocks with the male luer lock interlocking member 138. It will be understood that any type of conical, threaded, L-shaped slot and pin or other interlocking luer locks may be employed. The locking of interlocking members 128 and 138 advantageously prevents relative movement and slippage of the inner and outer members during passage through tissue in the selected body pathway.

When so assembled, the pointed shaft distal end 118 extends distally from the sheath distal end 132. In use, the handle 114 and the proximal sheath interlocking member 138 are grasped and manipulated to advance the nested shaft 116 and sheath 136 through a tissue pathway from a skin incision to dispose the exposed shaft distal end at the tissue test site 160 as in FIG. 2. Any of the insertion pathways disclosed in the prior art, particularly for placement of neural stimulation leads, may be followed.

Another embodiment of an introducer 210 for a neural stimulation lead is depicted in FIGS. 5 and 6 and comprises an inner member 212 and an outer member 230.

The inner member 212 comprises a proximal handle 214 and an elongated shaft 216 extending from a shaft distal end 218 through a shaft length to a shaft proximal end 220 coupled to and extending proximally from handle 214. In this embodiment, the shaft 216 comprises an elongated, electrically conductive, hollow needle having a tissue penetrating tip at the shaft distal end 218 although it will be understood that the shaft 216 may take other forms as noted above and may be only partly conductive along its length. A shaft lumen 246 that may be employed to deliver fluids or receive a guide wire extends from a proximal lumen end side opening at the shaft proximal end 220 to a distal lumen end opening 248 along the shaft distal end 218. The handle 214 is preferably non-conductive or has a non-conductive exterior surface coating. An electrically insulating coating may optionally be applied along an intermediate portion of the shaft 216 extending from a point near the shaft distal end 218 to the handle 214.

The outer member 230 of FIGS. 5 and 6 extends from an sheath distal end 232 through an outer length to a sheath or outer member proximal end 234 and is formed of an elongated sheath 236 coupled with a proximal sheath interlocking member 238 at the outer member proximal end 234. The sheath 236 may extend through the proximal sheath interlocking member 238 to or near the outer member proximal end 234. The elongated sheath 232 encloses an outer member or sheath lumen 240 extending between the outer member distal and proximal ends 232 and 234. In this embodiment, the sheath 236 is formed of an elongated, thin wall, electrically nonconductive, tubing that may or may not be tapered at the sheath distal end 232 to effect dilation of tissue that it is advanced through. A wing 244 extends across the sheath interlocking member 238, and two lines of perforations 242, disposed 180° apart around the circumference, extend along the length of the outer member 230 between the outer member distal and proximal ends 232 and 234. The lines of perforation weaken the outer member 230 so that it is splittable or capable of being torn apart along its length by grasping the opposed extensions of the wing 244 and pulling to split the outer member 230 into two parts along the lines 242 to facilitate removal from an electrical medical lead extending through the sheath lumen 240.

As shown in FIGS. 5 and 6, the inner member handle 214 is preferably ergonomically shaped to be gripped with one hand to advantageously facilitate directional control of advancement of the shaft and outer member body through tissue. The inner member handle 214 is shaped having opposed major surface area sides 250 and 252 joined by smaller surface area sides 254 and 256 and proximal and distal ends 258 and 260. The opposed major surface area sides 250 and 252 are shaped with complementary, generally concave surfaces adapted to be gripped between the fingers. Exemplary gripping surfaces 262, 264, 266 that may be embossed or ribbed or textured and/or contoured to enhance gripping and reduce the possibility of slipping are schematically depicted on major surface area side 250. It will be understood that the type, number, area, and placement of each such gripping surface 262, 264, 266 may be varied as determined to be efficacious, and that similar gripping surfaces may be provided on major surface area side 252.

The shaft proximal end 220 is electrically coupled to or forms an electrical connector 222 extending proximally from (in this embodiment) the nonconductive handle 214 through handle proximal end 258. In this embodiment, the shaft 216 is not curved within the inner member handle 214 and exits through a curved proximal portion of the major surface area side 250 near the handle proximal end 258. Again, the electrical connector 222 may simply be the electrically conductive exterior circumferential surface of the shaft 216 extending proximally from the handle proximal end 258 to the shaft proximal end 220. As shown in FIG. 2, an electrical cable 52 may be provided extending from the pulse generator output circuitry of the external neural test stimulator 50 to a cable connector 54, e.g., an alligator clip type connector, that may be attached to the connector 222 during application of the test stimuli. The test stimuli are conducted through the length of the inner member shaft 216 and applied to the tissue at the tissue test site 260 by the electrically conductive exposed shaft distal end 218 in contact therewith.

As shown in FIG. 6, the inner member shaft 216 is sized in relation to the sheath lumen 240 so that it may be extended therethrough, until the outer member proximal end 234 abuts the handle 214, to form the introducer 210. The inner member handle 214 and the outer member sheath 236 are advantageously coupled together and decoupled through manipulation of interlocking members at the inner member handle 214 and the outer member proximal end 234. In this embodiment, the outer member interlocking member 238 preferably comprises a female luer lock, and the inner member handle 214 incorporates a male luer lock handle interlocking member 228 extending distally of handle distal end 260 that is received within and interlocks with the female luer lock interlocking member 238. It will be understood that any type of conical, threaded, L-shaped slot and pin or other interlocking luer locks may be employed. The locking of interlocking members 228 and 238 advantageously prevents relative movement and slippage of the inner and outer members during passage through tissue in the selected body pathway.

When so assembled, the pointed shaft distal end 218 extends distally from the sheath distal end 232. In use, the handle 214 and the proximal sheath interlocking member 238 are grasped and manipulated to advance the nested shaft 216 and sheath 236 through a tissue pathway from a skin incision to dispose the exposed shaft distal end at the tissue test site 260 as in FIG. 2. Any of the insertion pathways disclosed in the prior art, particularly for placement of neural stimulation leads, may be followed.

Various ways of enhancing the interlocking force of the interlocking mechanisms securing the outer member to the inner member to resist inadvertent separation are contemplated. FIGS. 7 and 8 illustrate that the dimensions of an interlocking male pin 70 and female bore 80 of a luer connector are selected to provide an interference fit that increases separation resistance. The female bore opening 82 is preferably tapered to ease alignment and insertion of the male pin 70, and the interference fit diameters of the male pin 70 and female bore 80 enhance the interlocking force when the male pin 70 is fitted into the female member 80. The surface areas of the male pin 70 and female bore 80 may also be textured or ribbed with ribs 72 and/or 84 as shown in FIGS. 9 and 10.

A variety of other interlocking mechanisms are contemplated for interlocking the outer member connector with the inner member handle wherein a handle interlocking member comprises first and second handle interlocking elements and an outer member interlocking member comprises respective first and second outer member interlocking elements. The first outer member interlocking element may be adapted to engage the first handle interlocking element and the second outer member interlocking element may be adapted to engage the second handle interlocking element to affix the outer member proximal end to the handle distal end in a predetermined orientation that effects such an alignment.

An alternative clip and wing mechanism providing a handle interlocking member 328 for coupling an outer member with an inner member of an introducer of the types described herein is depicted in regard to introducer 310 depicted in FIGS. 11 and 12. In FIG. 11, the outer member 330 is positioned to be disposed over the inner member shaft 316 to be locked to the inner member handle 314. The inner member shaft 316 and handle 314 may incorporate any of the features of the various embodiments of the introducer 310 described herein and alternatives thereto. The outer member 330 depicted in FIGS. 11 and 12 includes an outer member sheath 336 enclosing a sheath lumen 340 extending between outer member proximal and distal ends 334 and 332, respectively, with the proximal wing 344 extending laterally of the outer member proximal end 334.

The outer member 330 therefore may take the form of any of the above-described outer members 130, 230 without a luer lock but including the wing 144, 244 and optionally including lines of perforations 142, 242.

In this embodiment, the wing 344 also functions as an outer member interlocking member, and the wing ends function as outer member interlocking elements 370 and 372, and the handle interlocking member 328 comprises clips 380 and 382 that are mounted to the handle distal end 360 to engage the respective outer member interlocking elements 370 and 372.

In use, the wing ends of wing 344 are clipped into clips 380 and 382 extending distally of the handle distal end 360 to provide secure engagement. The clips 380 and 382 therefore comprise first and second handle interlocking elements that engage the outer member interlocking member or wing 344 that comprises respective first and second outer member interlocking elements or wings. The inner member shaft 316 extends through the outer member lumen 340 of sheath 336 to electrically expose a distal tip 318 of the inner member shaft 316 distal to sheath distal end 338 during advancement through a tissue pathway. As described above with respect to FIG. 2, test stimuli emitted by an external neural test stimulator 50 is applied to an inner member connector extension of the inner member shaft extending proximally of the inner member handle 314, and the stimuli are conducted through the shaft and to tissue at tissue test site 60 proximate the exposed shaft distal end 318.

A further alternative interlocking mechanism for interlocking an outer member 430 with an inner member 412 of an introducer 410 of the types described herein is depicted in FIGS. 13 and 14. In FIG. 13, the outer member 430 is positioned to be disposed over the inner member shaft 416 to be locked into a handle interlocking member 428 at the distal end 460 of the inner member handle 414. The inner member shaft 416 and handle 414 may incorporate any of the features of the various embodiments of the introducer described herein and alternatives thereto. The outer member 430 depicted in FIGS. 13 and 14 includes an outer member sheath 436 enclosing a sheath lumen 440 extending between outer member proximal and distal ends 434 and 432, respectively. The outer member 430 depicted in FIGS. 13 and 14 may take the form of any of the outer members 30, 130, and 230 without a luer lock but including a proximal lock bar 444 (that may be proximal to an optional wing 144, 244 and include lines of perforations 142, 242).

The proximal lock bar 444 functions as an outer member interlocking member and comprises opposed outer member interlocking elements 470 and 472. The inner member handle 414 is shaped at its distal end 460 with distally extending clip-shaped handle interlocking elements 480 and 482 that define a slot and a cavity that receives the opposed outer member interlocking elements 470 and 472.

In use, the proximal lock bar 444 is inserted through the slot between the opposed outer member interlocking elements 470 and 472 as shown in FIG. 13 and then rotated 90° within the cavity as shown in FIG. 14 to provide secure engagement with the handle interlocking elements 480 and 482. As shown in FIG. 14, the inner member shaft 416 extends through the outer member lumen 440 of outer member sheath 436 and distal to sheath distal end 432 to electrically expose a distal tip 418 of the inner member shaft 416 during advancement through a tissue pathway. As described above with respect to FIG. 2, test stimuli emitted by an external neural test stimulator 50 is applied to an inner member connector extension of the inner member shaft 416 extending proximally of the inner member handle, and the stimuli are conducted through the shaft 416 and to tissue at tissue test site 60 proximate the exposed shaft distal end 418.

A still further alternative interlocking mechanism for interlocking an outer member 530 with an inner member 512 of an introducer 510 of the types described herein is depicted in FIGS. 15 and 16. The inner member shaft 516 and handle 514 may incorporate any of the features of the various embodiments of the introducer described herein and alternatives thereto. However, in this introducer embodiment, the outer member 530 is provided with an outer member connector 538 that conforms to and interlocks with the distal end 560 of the inner member handle 514 to form a distal extension of the inner member handle 514, the combination preferably presenting an ergonomic shape with opposed gripping surfaces as depicted in FIG. 16. In FIG. 15, the outer member 530 is positioned to be disposed over the inner member shaft 516 to be locked to the inner member handle 514. The outer member 530 depicted in FIGS. 15 and 16 includes an outer member sheath 536 enclosing a sheath lumen 540 extending between outer member proximal and distal ends 534 and 532, respectively. The outer member sheath 536 may extend through the outer member connector 538 so that the inner member shaft 516 may be inserted through the sheath lumen 540 as depicted in FIG. 15.

A variety of interlocking mechanisms are contemplated for interlocking the outer member connector 538 with the inner member handle 514 in a proper alignment of their opposed major surface area and minor sides. A handle interlocking member that comprise first and second handle interlocking elements can be provided to engage an outer member interlocking member that comprises respective first and second outer member interlocking elements. The first outer member interlocking element may be adapted to engage the first handle interlocking element and the second outer member interlocking element may be adapted to engage the second handle interlocking element to affix the outer member proximal end to the handle distal end in a predetermined orientation that effects such an alignment.

One preferred form of such a suitable interlocking elements illustrated in FIGS. 15 and 16 comprises a handle interlocking member 528 comprising a pair of male pins 580, 582 extending distally from the handle distal end 560 and an outer member interlocking member 538 comprising a pair of female sockets or bores 570, 572 extending distally into the outer member proximal end 534. As illustrated, the male pins 580, 582 extend distally from the handle distal end 560 generally in parallel with and on either side of the inner member shaft 516, and the mating pair of female bores 570, 572 extend from the sheath or outer member proximal end 534 into the connector 538 generally in parallel with and on either side of the outer member sheath 536. The pins 580, 582 and bores 570, 572 are sized in length to fully seat the pins 580, 582 in the bores 570, 572 and sized in diameter and/or surface treated as described with respect to FIGS. 7-10 to provide an interference fit frictional engagement along their respective lengths. It will be understood that the arrangement of the pins 580, 582 and bores 570, 572 may be reversed such that the pins extend proximally from the connector proximal end into mating bores extending proximally from the handle distal end 560 into the handle 514. Alternatively, if only one pin 570 or 572 and bore 580 or 582 is so reversed, the reversal would ensure that the outer sheath connector 538 is oriented properly with the handle 514 when the pins 580, 582 are fully seated.

As shown in FIG. 16, the inner member shaft 516 extends through the outer member lumen 540 to electrically expose a distal tip 518 of the inner member shaft 516 distal to the sheath distal end 532 during advancement through a tissue pathway. As described above with respect to FIG. 2, test stimuli emitted by an external neural test stimulator 50 is applied to an inner member connector extension 522 of the inner member shaft 516 extending proximally of the inner member handle 514, and the stimuli are conducted through the shaft 516 and to tissue at tissue test site 60 proximate the exposed shaft distal end 518.

A further alternative ergonomic handle of an inner member 612 adapted to be received in the sheath lumen of an outer member 630 of a further embodiment introducer 610 is depicted in FIGS. 17 and 18. The inner member 612 includes an elongated conductive shaft 616 extending through and proximally and distally of handle 614. The inner member shaft 616 is depicted extending through the lumen of an outer member 630 including an outer member proximal connector 638 and a distally extending outer member sheath 636 to dispose the shaft distal end 618 extending distally of the sheath distal end 632. As depicted, the elongated inner member shaft 616 comprises a hollow needle terminating in a distal lumen end opening, for example that corresponds to needle shaft 216 of FIGS. 5 and 6. The outer member 630 depicted in FIGS. 17 and 18 includes an outer member sheath 636 enclosing a sheath lumen 640 extending between outer member proximal and distal ends 634 and 632, respectively. The outer member 630 substantially corresponds to outer member 230 of FIGS. 5 and 6, and the interlocking members take the form of interlocking members 228, 238, simply for example.

In this embodiment, a resilient protective cap 680 is depicted placed over the connector extension 622 of the inner member shaft 616 extending proximally of the inner member handle 614 to shaft proximal end 620 to blunt the shaft proximal end 620 and facilitate handling and avoid penetrating a surgical glove. A bore 682 is formed in the protective cap to receive the extension 622, and the protective cap may be shaped to have a flat 684 as shown in broken lines in FIG. 17.

The manipulation of the inner member handle of FIG. 17 with the protective cap 680 in place during advancement of the inner member and an outer member through tissue is depicted in FIG. 18. The protective cap 680 placed over the shaft proximal end 620 may be somewhat resilient rubber and spherical or button shaped.

In the depicted embodiment, the outer member 630 takes the form of the outer member 230, and the interlocking members of handle 614 and outer member connector 638 take the form of interlocking members 228, 238, simply for example. It will be understood that a similar protective cap 680 may be placed over any of the exposed proximal shaft ends extending from the inner member handles of the inner members of any of the introducer embodiments disclosed herein.

Moreover, the protective cap 680 can be shaped to provide ergonomic gripping surfaces in the manner of the handle 514 of the inner member 512 of FIGS. 15 and 16. For example, a protective cap enlarged to provide opposed major surface areas bounded by opposed sides and ends, e.g., shaped in the manner of handles 214 or 514, may be provided with a cavity in its distal end dimensioned and shaped to receive and encase the proximally extending extension forming connector 22 terminating in the shaft proximal end 20 of shaft 16 and a proximal portion or all of the handle 14 of the inner member 30 of FIGS. 1 and 2. Such a cap would be employed during the steps of forming the tissue pathway to the target stimulation site and then removed to conduct the stimulation test as depicted in FIG. 2 and described above.

The introducer 710 depicted in FIGS. 19 and 20 redirects the proximal portion of the inner member shaft 716 extending through the inner member handle 714 to minimize interference in handling and manipulating the inner member handle 714. The inner member shaft 716 is extended either toward the side of the handle proximal end to exit therethrough and form the connector extension 722 or at any selected angle toward and through one of the handle sides to form the alternate connector extension 722′, both alternates shown in FIGS. 19 and 20. As shown in FIG. 20, the handle 714 may therefore be manipulated without touching the connector extension 722 or 722′. As described above with respect to FIG. 2, test stimuli emitted by an external neural test stimulator 50 is applied to an inner member connector extension 722 or 722′ of the inner member shaft 716 extending proximally of the inner member handle 714, and the stimuli are conducted through the shaft 716 and to tissue at tissue test site 60 proximate the exposed shaft distal end 718.

In the embodiment depicted in FIGS. 19 and 20, the outer member 730 takes the form of the outer member 630 of FIGS. 17 and 18, employing the interlocking members described above, simply for example. The outer member 730 includes an outer member sheath 736 enclosing a sheath lumen 740 extending between outer member proximal and distal ends 734 and 732, respectively.

Alternative configurations of the inner member electrical connector suitable for use in the embodiments of the introducer of the present invention, wherein the inner member shaft does not extend outward of the inner member handle to form the connector extension, are depicted in FIGS. 21 and 22. In the embodiments depicted in FIGS. 21 and 22, the interlocking members of the inner and outer members take the form of interlocking members 228, 238, simply as a suitable example.

More specifically, in FIG. 21, the introducer 810 comprises an outer member 830 and an inner member 812 comprising a handle 814 and an elongated solid needle shaft 816 extending from a shaft proximal end 820 embedded within handle 814 to a distal end 818. In this embodiment, the handle 814 is divided into two parts by a slot 880 that exposes a proximal section of the shaft 816 to function as the electrical connector 822. It will be understood that the slot 880 is sized to enable attachment of a cable connector to the electrical connector 822, and that the slot 880 may not necessarily extend all the way across and thereby split the handle 814. The outer member 830 takes the form of the outer member 630 of FIGS. 17 and 18, employing the interlocking members described above, simply for example. The outer member 830 includes an outer member sheath 836 enclosing a sheath lumen 840 extending between outer member proximal and distal ends 834 and 832, respectively.

In FIG. 22, the introducer 910 comprises an outer member 930 and an inner member 912 comprising a handle 914 and an elongated solid needle shaft 916 extending from a shaft proximal end 920 embedded within handle 914 to a distal end 918. In this embodiment, a port 980 is formed extending through the handle 914 that exposes a proximal section of the shaft 916 to function as the electrical connector 922. It will be understood that the slot 980 is sized to enable attachment of a cable connector to the electrical connector 922 and may not extend all the way through the handle 914. The outer member 930 takes the form of the outer member 630 of FIGS. 17 and 18, employing the interlocking members described above, simply for example. The outer member 930 includes an outer member sheath 936 enclosing a sheath lumen 940 extending between outer member proximal and distal ends 934 and 932, respectively.

In use of either introducer 810 or 910, a cable connector, e.g., the alligator clip 54 of cable 52 of FIG. 2, can be extended through the slot 880 or port 980 into engagement with the exposed connector 822 or 922. As described above with respect to FIG. 2, test stimuli emitted by an external neural test stimulator 50 is applied to the exposed connector 822 or 922 of inner member shaft 816 or 916, and the stimuli are conducted through the shaft 816 or 916 and to tissue at tissue test site 60 proximate the exposed shaft distal end 818 or 918.

An alternative electrical connector that may be incorporated into any of the embodiments of the inner member handle in substitution for the inner member connector extension of the inner member shaft or the slot 880 or port 980 is shown in FIG. 23. In this variation, the outer member 1030 is schematically depicted and may take any of the forms of outer members described herein. The outer member 1030 comprises an elongated sheath 1036 enclosing a sheath lumen 1040 and extending between a sheath or outer member distal end 1032 and a sheath or outer member proximal end 1034. The interlocking members of the inner and outer members 1012 and 1030 take any form.

In this variation of an inner member 1012, the inner member shaft 1016 extends into the proximal handle 1014 and is electrically connected to any suitable plug-in electrical connector molded into the handle 1014 to be accessed through a side or proximal end of the handle 1014. For example, a port 1080 is formed in the handle proximal end 1058 to access a female electrical connector socket 1066 as depicted in FIG. 23. The port 1080 receives a male connector 56 (substituted for the alligator clip 54) that is coupled through cable 52 to the external neural test stimulator 50. The proximal end of the inner member shaft 1016 is either directly coupled to the connector socket 1066 that receives the connector pin 58 or is connected thereto through an intermediate electrical wire or connector structure anywhere within the handle 1014. The depicted male connector 54 has a second pin 68 that fits into a second terminal bore or socket 1068 within port 1080 that may or may not be electrically coupled to the shaft 1016 for redundancy but provides stability to the connection. As described above with respect to FIG. 2, test stimuli emitted by an external neural test stimulator 50 is applied through the pin 58 and socket 1068 to the inner member shaft 1016, and the stimuli are conducted through the shaft 1016 and to tissue at tissue test site 60 proximate the exposed shaft distal end. It will be understood that the arrangement of the pins 58, 68, and the sockets 1066, 1068 may be reversed such that pins extend into the port and the connector 56 incorporates the mating sockets.

The preferred embodiments described above provide unipolar electrical stimulation through delivery of the test stimuli through the electrode disposed at the test site and a return electrode on a pad placed against the patient's skin. Thus, the connectors 54 and 1080 may comprise only a single pin 58 and bore or socket 1020 conforming to any single pole connector standard.

Such a further variation on the embodiment of FIG. 23 is depicted in FIG. 24. The port 1080′ is formed of a circular bore or port into the proximal end 1058′ of the modified handle 1014′ of the modified inner member 1012′. The shaft 1016 is extended into the port 1080′ such the exposed section 1022 and proximal end 1020 form a male connector pin. The female cable connector 56′ is cylindrical to fit within the port 1080′ and incorporates a bore or socket that receives the male connector pin comprising the exposed section 1022 and proximal end 1020.

As noted above, any of the above-described inner member shafts may be formed of a non-conductive material with at least one conductor extending between the proximal connector and a conductive distal tip functioning as the stimulation electrode. For example, a conductive strip and an electrode surface may be printed or plated on the exterior surface of the inner member shaft and exposed distal tip end to form the conductor and distal electrode. Or a conductive distal tip attached to the non-conductive shaft body may be coupled through a conductor plated or printed or adhered onto or within the non-conductive shaft body extending proximally to the handle connector.

It will also be understood that two spaced-apart electrodes may be provided at the exposed distal end of the inner member shaft that are coupled through conductors extending the length of the inner member shaft to two connector pins of any of the types described above or to the sockets 1022 and 1068 depicted in FIG. 23, for example, to provide bipolar stimulation at the tissue test site 60. Again, conductive strips and electrode surfaces may be printed or plated on the exterior surface of the inner member shaft to form each conductor and the spaced-apart electrodes on the distal end 1018. Alternatively, the distal end 1018 may be formed of spaced-apart ring and tip electrodes attached to the non-conductive shaft body and coupled through a pair of conductors plated or printed on or adhered onto or within the non-conductive shaft body extending proximally to the handle connector and from there to the two connector sockets 1022 and 1068 of connector 1080. The bipolar stimulation would be applied from the test stimulator through the connector pins 58 and 68 in a manner well known in the art.

Advantageously, the minimally invasive introducer embodiments and procedures minimize patient trauma and procedure time while ensuring safe and reliable introduction of neural stimulation leads.

1. An introducer (10; 110; 210; 310; 410; 510; 610; 710; 810; 910; 1010) adapted to be gripped to form a tissue pathway from a skin incision to a stimulation site (60) in a patient's body and to facilitate advancement of an electrical medical lead through the pathway comprising:
an elongated outer member (30; 130; 230; 330; 430; 530; 630; 730; 830; 930; 1030) having an elongated sheath (36; 136; 236; 336; 436; 536; 636; 736; 836; 936; 1036) enclosing a sheath lumen (40; 140; 240; 340; 440; 540; 640; 740; 940; 940; 1040) extending between an outer member proximal end (34; 134; 234; 334; 434; 534; 634; 734; 834; 934; 1034) and an outer member distal end (32; 132; 232; 332; 432; 532; 632; 732; 832; 932; 1032);
an elongated inner member (12; 112; 212; 312; 412; 512; 612; 712; 812; 912; 1012, 1012′) comprising a proximal inner member handle (14; 114; 214; 314; 414; 514; 614; 714; 814; 914; 1014, 1014′) having gripping surfaces that are non-conductive and adapted to be gripped in use of the introducer and an electrically conductive elongated shaft (16; 116; 216; 316; 416; 516; 616; 716; 816; 916; 1016) extending proximally into the handle from a handle distal end (160; 260; 360; 460; 560; 1060), the shaft extending between a shaft proximal end (20; 120; 220; 320; 420; 520; 620; 720; 720′ 820; 920; 1020) and a shaft distal end (18; 118; 218; 318; 418; 518; 618; 718; 818; 918; 1018) and sized to fit through the sheath lumen to extend the shaft distal end distally from the sheath distal end to thereby expose the shaft distal end and to enable advancement of the shaft and sheath through a tissue pathway by manipulation of the handle gripping surfaces outside the patient's body; and characterized by:
an electrical connector (22; 122; 222; 322; 422; 522; 622; 722; 822; 922; 1022) coupled to the shaft proximal end or comprising a section of the elongated shaft and supported by the handle proximal to the handle distal end, the electrical connector adapted to be coupled to an external test stimulator (50) operable to generate test stimuli conducted through the electrically conductive shaft and applied to nerves and tissue proximate the exposed shaft distal end.

2. The introducer of claim 1, wherein the outer member sheath is non-conductive to electrically insulate the shaft proximal to the exposed shaft distal end.

3. The introducer of claim 2, wherein the outer member sheath is formed with perforations (142, 242) to facilitate splitting and removal of the outer member following disposition of the one or more lead electrode proximate to body nerves or tissue to be electrically stimulated.

4. The introducer of claims 3, wherein:
the inner member comprises or functions as one of a stiffening stylet, an obturator, a solid shaft needle, and a hollow core needle adapted to enable advancement of a guide wire through the needle lumen; and
the outer member may comprises or functions as one or more of a cannula or a dilator having a sheath lumen sized to enable advancement of the electrical medical lead through the sheath lumen by itself or over a guidewire introduced through the sheath lumen.

5. The introducer of claim 1, wherein the gripping surfaces of the inner member handle are ergonomically shaped to be gripped with one hand to advantageously facilitate directional control of advancement of the inner member shaft and outer member body through tissue.

6. The introducer of claim 5, wherein the inner member handle is shaped having opposed major surface area sides (150, 152; 250, 252; 650; 750) joined by smaller surface area sides (154, 156; 254, 256; 754, 756), a handle proximal end (158; 258), and the handle distal end (160; 260; 360; 460; 560; 1060), and the opposed major surface area sides are shaped with complementary, generally concave surfaces adapted to be gripped between the fingers.

7. The introducer of claim 5, wherein:
the inner member handle is shaped having opposed major surface area sides joined by smaller surface area lateral sides, a handle proximal end, and the handle distal end; and
the inner member shaft extends into the handle distal end, through the inner member handle, and out of one of a handle major surface area side (254), a handle lateral side (754), or the handle proximal end (158; 358; 558; 658; 758) to expose a proximal section of the inner member shaft to constitute or be coupled with the electrical connector.

8. The introducer of claim 1, wherein:
the inner member handle is shaped having opposed major surface area sides joined by smaller surface area lateral sides, a handle proximal end and the handle distal end; and
the inner member shaft extends into the handle distal end, through the inner member handle, and out of one of a handle major surface area side, a handle lateral side, or the handle proximal end to expose a proximal section of the inner member shaft to constitute or be coupled with the electrical connector.

9. The introducer of claim 8, wherein the exposed proximal section (722, 722′) of the inner member shaft (716) extends away from the handle (714) at a point and in a direction that minimizes interference with manipulation of the handle and application of directional control to the inner member shaft and outer member sheath.

10. The introducer of claim 8, further comprising a disposable resilient cap (680) shaped to fit over the exposed proximal section of the inner member shaft (616) constituting the electrical connector (622) to blunt the shaft proximal end (620) and minimize interference with manipulation of the handle and application of directional control to the inner member shaft and outer member sheath.

11. The introducer of claim 1, wherein:
the inner member handle (814; 914; 1014′) is shaped having opposed major surface area sides joined by smaller surface area lateral sides, a handle proximal end, and the handle distal end (858; 958; 1058′), and is formed with a connector access port (880; 980; 1080′);
the inner member shaft (816; 916; 1016) extends into the handle distal end and through at least a portion of the inner member handle and into or though the connector access port to expose the electrical connector (822; 922; 1022), whereby an electrical connection of the electrical connector with a test stimulator is effected through the connector access port.

12. The introducer of claim 11, wherein the connector access port (822) comprises a slot extending through the handle major surface area sides and laterally between the lateral sides exposing a section of the shaft to function as the electrical connector.

13. The introducer of claim 11, wherein the connector access port (980) extends laterally through the handle major surface area sides between the lateral sides exposing a section of the shaft to function as the electrical connector.

14. The introducer of claim 1, wherein:
the inner member handle (914; 1014; 1014′) is shaped having opposed major surface area sides joined by smaller surface area lateral sides, a handle proximal end (958; 1058; 1058′), and the handle distal end, and is formed with a connector access port (980; 1080; 1080′);
the electrical connector (922; 1066; 1022) is disposed in the connector access port (980; 1080; 1080′); and
the inner member shaft (916; 1016) extends into the handle distal end and through at least a portion of the inner member handle and is electrically connected to or constitutes the electrical connector disposed in the connector access port, whereby an electrical connection of the electrical connector with a test stimulator is effected through the connector access port.

15. The introducer of claim 14, wherein the connector access port is disposed in the handle proximal end (1058′).

16. The introducer of claim 15, wherein the shaft distal end 1020 extends into the connector access port (1080′) to form an electrical connector pin (1022).

17. The introducer of claim 1, wherein:
the inner member handle (14; 114; 214; 314; 414; 514) comprises a handle interlocking member (28; 128; 228; 328; 428; 528) substantially at the junction of the inner member shaft (16; 116; 216; 316; 416; 516) with the inner member handle (14; 114; 214; 314; 414; 514); and
the outer member proximal end (34; 134; 234; 334; 434; 534) comprises an outer member interlocking member (38; 138; 238; 344; 444; 538) configured to interlock with the handle interlocking member.

18. The introducer of claim 17, wherein:
the handle interlocking member (328; 428; 528) comprises first and second handle interlocking elements (380, 382; 480, 482; 580, 582); and the
outer member interlocking member (344; 444 538) comprises a first outer member interlocking element (370; 470; 570) adapted to engage the first handle interlocking element (380; 480; 580) and a second outer member interlocking element (372; 472; 572) adapted to engage the second handle interlocking element (382; 482; 582) to affix the outer member proximal end to the handle distal end in a predetermined orientation.

19. The introducer of claim 1, further comprising a protective cap (680) shaped to receive the shaft proximal end (620) to facilitate handling of the introducer.

20. The introducer of the preceding claim 1, wherein the sheath lumen is sized to facilitate advancement of an electrical medical lead through the sheath lumen following removal of the inner member to dispose one or more distal lead electrode proximate to body nerves or tissue to be electrically stimulated by a stimulator coupled to a proximal connector of the electrical medical lead.
